(19) United States
(12) Patent Application Publication
(10) Pub. No.: US 2019/0083333 A1
(43) Pub. Date: Mar. 21, 2019

(54) WAIST BELT AND BELTED ABSORBENT ARTICLE

(71) Applicant: SCA HYGIENE PRODUCTS AB, Goteborg (SE)

(72) Inventors: Maria SWEDBERG, Göteborg (SE); Anders SILFVERSTRAND, Göteborg (SE); Åsa JOHANSSON, Göteborg (SE)

(73) Assignee: SCA HYGIENE PRODUCTS AB, Göteborg (SE)

(21) Appl. No.: 15/760,884

(22) PCT Filed: Sep. 18, 2015
(86) PCT No.: PCT/SE2015/050975

§ 371 Date: Mar. 16, 2018

(51) Int. Cl. A61F 13/56; A61F 13/62; A61F 13/49

(52) U.S. Cl. CPC A61F 13/5633; A61F 2013/49092; A61F 13/49011; A61F 13/622

(57) ABSTRACT

A waist belt for an absorbent article has an inner surface facing the wearer during use and an outer surface facing away from the wearer during use. The belt includes a first belt end section provided with a fastening component and a second belt end section provided with a mating fastening component. Each one of the fastening component and the mating fastening component includes a first mechanical connector structure and a second mechanical connector structure. Each mechanical connector structure is capable of forming a mechanical interconnection with the other mechanical connector structure, and is incapable of forming a mechanical interconnection with an identical structure. Further, the fastening component and the mating fastening component are mechanically connectable via the first mechanical connector structure and the second mechanical connector structure. The disclosure also relates to a disposable article including a chassis and a waist belt.

### TECHNICAL FIELD

This disclosure relates a waist belt for an absorbent article. More particularly, the present disclosure relates to a waist belt for an absorbent article, wherein said belt comprises a first belt end section provided with a fastening component and a second belt end section provided with a mating fastening component. In addition, present disclosure relates to an absorbent article comprising a chassis and an integrated waist belt. Furthermore, the present disclosure relates to an absorbent article comprising a chassis and a waist belt, said waist belt being provided separate from said chassis. The belted absorbent article may be provided in various sizes.

### BACKGROUND

Absorbent articles in the form of disposable diapers are generally known in which the diaper is provided with a fastening system including a pair of fastening tabs secured to both sides of one end region of the diaper. The fastening tabs are intended to engage receiving means located on the other end region of the diaper. Such a diaper is generally placed on the wearer when the wearer is lying down. While the above-described fastening arrangement is satisfactory when applying diapers to a young child or baby, problems can arise when trying to fit an incontinence diaper to an adult wearer. This is particularly true for wearers who wish to apply the diaper to themselves, or for caregivers who receive a significant benefit from applying the diaper when the wearer is in a standing position. To address this issue, a belted absorbent article has been developed, which allows a wearer or caregiver to apply the diaper in a standing position. An example of such a belted absorbent article is described in U.S. Pat. No. 6,342,050, which is incorporated herein by reference. Typically, the absorbent article has a belt attached integrally with the chassis or the absorbent article. The belt may have two belt portions extending on either side of the rear end of the chassis or the absorbent section. The two belt portions are intended to be fastened around the waist of the wearer, whilst the front end of the absorbent section hangs down between the legs of the wearer. Once the belt portions have been joined together, the wearer can reach between his or her legs to draw up the absorbent section between the legs and to attach the free end of the absorbent section to the belt portion. Similarly, the article can be made so that the belt is fastened to the front portion of the product and is secured around the back of the wearer. In this case, the absorption section will be hanging down in the front and will be secured to the belt in the back. This type of product is particularly useful for caregivers who care for patients that may have dementia or the like. It should be noted that this type of configuration is not ideal for wearers who wish to apply the article themselves.

In other absorbent articles, the belt may form a separate part detachably attached to the chassis.

Typically, the belt is provided with special receiving surfaces designed to cooperate with hook elements. It is also common that the entire belt is manufactured from a material that can cooperate with the hook elements, wherein no special receiving surfaces are arranged on the belt.

### SUMMARY

An object of the present disclosure is to improve the fastening ability of the two belt end sections to each other so that the waist belt can be secured around the wearer of an absorbent article in a simple and effective manner. This object is at least partly achieved by the features of claim 1.

The disclosure concerns a waist belt for an absorbent article having an inner surface facing the wearer during use and an outer surface facing away from the wearer during use. The belt comprises a first belt end section provided with a fastening component and a second belt end section provided with a mating fastening component. Each one of the fastening component and the mating fastening component comprises a first mechanical connector structure and a second mechanical connector structure. Further, each mechanical connector structure being capable of forming a mechanical interconnection with said other mechanical connector structure, and being incapable of forming a mechanical interconnection with an identical structure, whereby said fastening component and said mating fastening component are mechanically connectable via said first mechanical connector structure and said second mechanical connector structure to form an interconnection between said first belt end section and said second belt end section for securing to each other around a wearer of the absorbent article.

In this manner, there is provided a more secure connection between the fastening component of the first belt end section and the mating fastening component of the second end belt section. In addition, by the provision that each fastening component and mating fastening component comprising first mechanical connector structure and second mechanical connector structure, as described above, it becomes possible to provide a high level of contact between the components. Furthermore, the mechanical interconnection provided by having two types of mechanical connector structures on each fastening component contributes to minimize the risks of having a poor fastening of the absorbent article around the wearer during use. As a poor fastening often results in a poor fit of the article and an increased risk of leakage, the example embodiments of the disclosure may also have a positive impact on overall fit of the article, whilst reducing the risk of leakage during use of the article.

Due to that the fastening component and the mating fastening component comprising both first mechanical connector structure and second mechanical connector structure, the peel strength and shear strength of the fastening connection is improved as the number and type of connection areas/points of the connection is increased in view of a belt end section comprising a hook material configured for attaching to a loop material of another belt end section. The increased number of connection areas/points also contributes to an increased flexibility of adjustment of the waist belt during use.

To this end, the increased number of connection areas/points may provide the option of using the connection points as various size indications, enabling the use to easily understand where to engage one of the belt end section to the other belt end section to obtain a comfort and secure belted configuration. In some example embodiments, the connection points or a number of the mechanical connector structures may be coloured to further improve the function of using the fastening component and/or the mating fastening component as various size indicators.

In addition, the example embodiments of the disclosure provides the advantage that less considerations is needed as to the type of material of the belt end sections of the waist belt because the interconnection is made between the first mechanical connector structure and the second mechanical connector structure of the components, and thus not between e.g. a hook material on one belt end section and a surface of the other belt end section. The latter often requires that the material of the belt end section acting as the receiving surface for the hook material is carefully selected in terms of fastening properties etc. Thus, the configuration of the fastening component and the mating fastening components may increase the design freedom of the material of the belt end sections.

For the same reasons, the configuration of the fastening component and the mating fastening component results in that the materials making up the mechanical interconnection can be selected almost independently on type of material of the belt end sections, thus more optimal materials of the fastening component and the mating fastening component may be selected, which e.g. will reduce the amount of fuzz on the mechanical connector structure during use of the waist belt, e.g. if the connector structure is a hook material, compared to a waist belt having a hook material on one belt end section to engage directly with a nonwoven material on the other belt end section.

Accordingly, there is provided a waist belt and belted article which is more pliable, more flexible and less stiff than current prior art belted articles, whilst maintaining a high level of wearer comfort.

The object of the present disclosure is also at least partly achieved by the features of an absorbent, wherein the absorbent article comprises a chassis and a waist belt according to any one of the features, examples or variants mentioned above with the respect to the waist belt. The chassis has first and second end portions and a central portion extending therebetween, and further includes a first covering layer having a first surface intended to face towards the user and a second surface intended to face away from the user in use. Moreover, the waist belt is attached to the chassis so that said first belt end section and second belt end section extends on each side of the first end portion of the chassis for securing to each other around a wearer of the article, and wherein said second end portion of the chassis includes a fastening device for securing the second end portion of the chassis to the belt.

The object of the present disclosure is also at least partly achieved by the features of an absorbent article, wherein the absorbent article comprises a chassis and a waist belt according to any one of the features, examples or variants mentioned above with the respect to the waist belt, and wherein said waist belt is provided separate from said chassis.

The object of the present disclosure is also at least partly achieved by the features of an extension belt including any one of the features, examples or variants mentioned above with the respect to the waist belt.

Further advantages are achieved by implementing one or several of the features of the dependent claims.

In the example embodiments, a fastening component thus typically comprises a first mechanical connector structure and a second mechanical connector structure, the first and second mechanical connector structures being two corresponding structures, each structure being capable of forming a mechanical interconnection with the other structure, and being incapable of forming a mechanical interconnection with an identical structure, whereby the fastening component is mechanically connectable to a mating fastening component, the mating fastening component comprising the first and second mechanical connector structures.

The terms “fastening component” and “mating fastening component” as used herein is generally a portion of material which potentially (if in contact with another fastening component or mating fastening component) may perform interconnection of the belt end section to the other belt end section by means of the first and second mechanical connector structures.

The mechanical connector structures herein refers to connector structures for mechanically interconnecting one of the belt end sections to another belt end sections, as is typically required to form continuous and closed waist belt when the article is to be used.

Mechanical connector structures have the advantage of providing a secure connection between themselves, whilst not being prone to unwanted connection to other materials, such as to the belt section material itself. Hence, use of mechanical connector structures provides an improved and reliable connection of the waist belt end section during use of the article, which is hitherto not provided by prior art articles.

Mechanical connector structures may often be of a type where the connection is accomplished by a pair of two different, corresponding interconnectable connector structures. The first connector structure is hence capable of forming a mechanical interconnection with a second connector structure, but not with another first connector structure (i.e. an identical structure). Similarly, the second connector structure is capable of forming a mechanical interconnection with the first connector structure, but not with another second connector structure (i.e. an identical structure). A typical example of such connector structures is a hook structure and a corresponding loop structure.

Typically, although not strictly required, the first and second connector structures being arranged in a pattern. It will be understood that the first and second mechanical connector structures are arranged on a first side of the fastening component. One of the first and second connector structures may comprise hooks, and the other of the first and the second connector structures may comprise loops.

Alternatively, one of said first and second connector structures may comprise a first type of hooks, and the other of the first and the second connector structures may comprises a second type of hooks, said first type of hooks having a different hook characteristics than said second type of hooks.

The first connector structure may be provided by a first connector material and the second connector structure may be provided by a second connector material.

According to example embodiments, the second connector material may be attached to the first connector material, preferably onto the first connector material.

According to example embodiments, the fastening component and the mating fastening component comprises a first connector material onto which intermittent pieces of second connector material are attached. The fastening component and the mating fastening component may comprise a first connector material onto which is attached a second connector material in which through holes are formed, such that the first connector structures of the first connector material is accessible via the through holes in the second connector material.

The fastening component and the mating fastening component may comprise a carrier material, onto which the first and/or second connector material is attached.

The fastening component and the mating fastening component may be a single connector material comprising a backing from which the connector structures extend.

The fastening component and the mating fastening component may display a repeated pattern for forming individual connectors.

The fastening component and the mating fastening component may each display a repeated pattern having a length and a width, preferably the length being a length with which the pattern repetition is evenly divisible.

It is proposed herein that each one of the fastening component and the mating fastening component of the belt end sections, is to comprise first and second mechanical connector structures. In accordance with the above, a waist belt and a belted article is provided which enables an improved closed configuration of the waist belt, since each one of the fastening component and the mating fastening component of comprising both first and second connector structures

To provide an interconnection, it is generally not required that the entire surface area formed by the first mechanical connector structure and/or the second mechanical connector structure of a fastening component is interconnected to a second and/or first mechanical connector structure of a mating fastening component. With mechanical fastening components, sufficient connection strength may be achieved already with relatively small interconnected areas of mechanical connector structures. Hence, to provide an interconnection, it may be sufficient that the fastening components are designed so as to enable interconnection between a portion of the first connector structure of the fastening component and a portion of a corresponding second structure of the mating fastening component, or vice versa.

Typically, said first mechanical connector structure and said second mechanical connector structure of said fastening component is arranged in a first pattern and the first mechanical connector structure and the second mechanical connector structure of said mating fastening component is arranged in a second pattern. Said first pattern and said second pattern are formed so that at least a portion of the first mechanical connector structure of said fastening component mirrors at least a portion of the second mechanical connector structure of said mating fastening component and at least a portion of the second mechanical connector structure of said fastening component mirrors at least a portion of the first mechanical connector structure of said mating fastening component. The term “pattern” here refers to an organised arrangement of at least one area comprising the first mechanical connector structure, and at least one area comprising the second mechanical connector structure, in contrast to the random arrangement as described in the above. Further, the term “mirror”, “mirroring” or “a mirror image” refers to the relative arrangement of the two mechanical connector structures in the first pattern and the two mechanical connector structures in the second pattern when said fastening component and said mating fastening component are joined so as to form a closed waist belt configuration. That is, the above provision typically refers to a mirroring of the portions when the belted article is formed into the pant-type configuration.

According to one example embodiment, said first mechanical connector structure and said second mechanical connector structure of said fastening component is arranged in a first pattern and the first mechanical connector structure and the second mechanical connector structure of said mating fastening component is arranged in a second pattern, said first pattern being a mirror image of said second pattern. As mentioned above, the term “mirror image” refers to the relative arrangement of the two mechanical connector structures in the first pattern and the two mechanical connector structures in the second pattern when said fastening component and said mating fastening component are joined so as to form the closed waist belt configuration. Typically, the entire first pattern is a mirror image of the entire second pattern. In other words, the arrangement of the first mechanical connector structure and the second mechanical connector structure of said fastening component is a mirror image of the arrangement of the first mechanical connector structure and the second mechanical connector structure of said mating fastening component.

In this manner, the first mechanical connector structure and the second mechanical connector structure of the fastening component and the first mechanical connector structure and the second mechanical connector structure of the mating fastening component are capable of interconnecting over the entire area of the two different connector structures, thereby increasing the interconnection between the belt end sections to a maximum.

Typically, although not strictly necessary, the extension and orientation of the fastening component on the first belt end section, as seen in the direction x and direction y, corresponds to the extension and orientation of the mating fastening component on the second belt end section, as seen in the direction x and direction y.

According to one example embodiment, said second belt end section comprises a plurality of mating fastening component arranged spaced apart on the outer surface of said second belt end section, each mating fastening component comprising first mechanical connector structure and second mechanical connector structure, so that said fastening component and any one of the plurality of said mating fastening components are mechanically connectable via said first mechanical connector structure and said second mechanical connector structure to form an interconnection between said belt end section and said second belt end section for securing to each other around a wearer of the absorbent article.

Hereby, the waist belt is provided with an increased level of adjustment and flexibility for the user of the article.

In certain variants, the fastening component and the mating fastening component may be different. In certain variants, the fastening component and the mating fastening component may be similar.

The first and the second mechanical connector structures may be randomly arranged on the fastening component and the mating fastening component. In this case, the first and second mechanical connector structures may be provided by a connector material on which first and second connector structures are formed in a random arrangement. For example, such a connector material could be a compound material designed to interconnect with an identical compound material, comprising randomly arranged first and second mechanical connector structures, e.g. randomly arranged hooks and loops protruding from a common backing material. Alternatively, the first and second mechanical connector structures per se may be arranged in an organised manner on a common backing material, but on a scale which will still result in a random arrangement as seen over the at least one connector.

Alternatively, at least one of the first and second connector structures may be arranged to form a pattern over the corresponding surface of the fastening component. With “a pattern” is meant herein an organised arrangement of at least one area comprising the first mechanical connector structure, and at least one area comprising the second mechanical connector structure, in contrast to the random arrangement as described in the above.

Such a pattern may be formed by various types of connector materials as will be described in the below. The pattern may be selected in various manners to provide sufficient likelihood of interconnection between the fastening component and the mating fastening component of the waist belt.

Optional, such a pattern of a fastening component may be adapted such that the pattern is mechanically connectable to an identical pattern surface, (i.e. provided with an identical pattern) of a mating fastening component.

In this disclosure, the term “identical” is meant herein something which is sufficiently identical considering the intended purpose of the features. Manufacturing tolerances or slight variations which do not affect the function of the feature is to be comprised in the term “identical”.

Alternatively, such a pattern of a fastening component may be adapted such that the pattern surface is mechanically connectable to a different pattern surface, (e.g. provided with a different pattern) of a mating fastening component.

That the end surfaces of the fastening components are mechanically connectable means that they may be connected, at least if the surface of a fastening component is provided with a specific orientation relative to the surface of mating fastening component.

The fastening component may define a longitudinal central axis (X), extending in parallel to the length fastening component, and centrally in relation to the width of the fastening component, wherein the pattern formed by the first and the second connector structures may be asymmetrical with respect to the longitudinal central axis (X).

Patterns which are asymmetrical with respect to the longitudinal central axis, may be designed so as to provide interconnections independently of the relative orientation of the fastening component and the mating fastening component.

The fastening component may define a transversal central axis (Y), extending in parallel to the width of the fastening component, and centrally in relation to the length of the fastening component, wherein the pattern formed by the first and the second connector structures is asymmetrical with respect to the transversal central axis (Y).

Patterns which are asymmetrical with respect to the transversal central axis, may be designed so as provide interconnections independently of the relative orientation of the fastening component and the mating fastening component.

The pattern formed by the first and the second connector structures may be such that at least a portion of the first structure on one side of the longitudinal central axis (X), mirrors a portion of the second structure on the other side of the longitudinal central axis (X), preferably all portions of the first structure on one side of the longitudinal central axis mirrors a portion of the second structure on the other side of the longitudinal central axis.

With (mating) fastening components comprising mirroring first and second structures over the longitudinal central axis may, interconnectivity between two similar fastening components and mating fastening components, regardless of their relative orientation may be achieved.

However, it is to be noted that the fastening component and the mating fastening component must necessarily not be identical in terms of shape and geometry, but that the width, length, pattern and/or shape of the fastening component may be different to the width, length, pattern and/or shape of the mating fastening component.

The pattern formed by the first and the second connector structures may be such that at least a portion of the first structure on one side of the transverse central axis (Y), mirrors a portion of the second structure on the other side of the transverse central axis (Y), preferably all portions of the first structure on one side of the transversal central axis (Y) mirrors a portion of the second structure on the other side of the transversal central axis (Y).

With fastening components and mating fastening components comprising mirroring first and second structures as seen over the transversal central axis, two similar fastening components and mating fastening components may be interconnected regardless of their orientation in relation to the transverse central axis.

In particular, at least one portion of the first structure, located on one side of the longitudinal central axis (X) and on one side of the transversal central axis (Y); may mirror a portion of the second structure on the other side of the longitudinal central axis (X) and a portion of the second structure on the other side of the transversal central axis (Y).

A fastening component (or mating fastening component) where at least one portion of the first structure mirrors a portion of the second structure over the longitudinal central axis (X), and a portion of the second structure over the transversal axis (Y), may be connected to another, identical mating fastening component, regardless of the relative orientation of the components as long as they meet upon fastening of the waist belt.

It may be that only one out of the first and the second connector structures is located on one side of the longitudinal central axis (X). This will be sufficient e.g. for forming e.g. a mirroring pattern as described in the above.

One of the first and the second connector structures may comprise hooks, and the other of the first and the second connector structures may comprise loops. Hook and loop materials providing sufficient strength and suppleness for the application intended herein are commercially available, e.g. under the trademark Velcro®.

Examples of suitable loop materials are knitted loop, brushed loop or a nonwoven loop materials. This type of materials are commercially available e.g. from 3M. Examples of suitable hook types are moulded hooks and textile hooks. The hooks can have different configurations, e.g. mushroom. Hook materials are commercially available e.g. from Velcro.

The fastening components and mating fastening components may each have a height of less than 2 mm, preferably less than 1 mm, most preferred less than 0.6 mm.

The first mechanical connector structure may be provided by a first connector material, and the second mechanical connector structure may be provided by a second connector material. For example, the first connector structure may be a loop material, and the second connector structure may be a hook material.

The fastening components and mating fastening components may be directly or indirectly attached to the waist belt end sections. For example, the fastening components and mating fastening components may be adhesively attached or ultra-sonic bonded to the surface of the belt end section. The first connector material may be attached to the second connector material, and the second connector material may be attached to the respective surface of the belt end section. In this case, the first connector material will be indirectly attached to the belt end section. Likewise, the second connector material may be directly or indirectly attached to the belt end section.

The fastening components and mating fastening components may comprise a carrier material, onto which the first and/or second connector material is attached, the carrier material being attached to the respective belt end sections. In this case, both fastening components and mating fastening components will be indirectly attached to the respective the belt end sections via the carrier. The carrier material may be directly or indirectly attached to the belt end sections.

In the fastening components and mating fastening components, the first connector structure and the second connector structure may be provided by a single continuous connector material.

Such a continuous connector material will hence be provided with both first and second connector structures. The continuous connector material could be a material where the first and second structures are randomly arranged, as mentioned in the above. The continuous connector material could also be a material where the first and second structures are arranged in a selected pattern.

The first connector material, the second connector material, the carrier material, or the single continuous connector material, respectively, may be band-shaped. Provision of the connector materials as band-shaped materials may be advantageous in view of manufacturing. Such band-shaped materials could in some alternatives be arranged over the full length of the belt end sections.

It has been realised, that with mechanical connectors, relatively small areas of interconnected connector structures are needed to provide sufficient connection strength. The shear force between the first connector and the second connector, when interconnected, reflects the strength of the interconnection.

Typically, said fastening component is arranged on the inner surface of said first belt end section and said mating fastening component is arranged on the outer surface of said second belt end section.

The first mechanical connector structure may be provided by a first connector material and said second mechanical connector structure may be provided by a second connector material. Said second connector material may be attached to said first connector material, preferably onto said first connector material.

In some example embodiments, any one of the first belt end section and the second belt end section comprises an elasticised region.

In some example embodiments, the waist belt is an extension belt product for an absorbent article.

In some example embodiments, an intermediate belt section is arranged between said first belt end section and said second end belt section to form a waist belt intended for use with an absorbent article.

According to one embodiment, the width in the direction x and the length in the direction y of the fastening component are substantially equal to the width in the direction x and the length in the direction y of the mating fastening component.

The term “fastening component” refers to the fastening elements that define an area of refastenable attachment. The fastening components enable refastening of the waist belt to reconfigure/adjust the width of the waist belt into a closed configuration until the fastening components are separated. Analogously, the term “mating fastening component” refers to a fastening elements that define an area of refastenable attachment, and which is configured to connect and cooperate with another fastening component. The mating fastening components enable refastening of the waist belt to reconfigure/adjust the width of the waist belt into a closed configuration until the fastening components are separated.

Thus, the term “mating” is used herein to define a pair of fastening component arranged and configured to cooperate and connect to form the waist belt configuration. Accordingly, the term “mating” refers to the arrangement of the mating fastening component as seen when the waist belt is in its closed.

Further features of, and advantages with, the example embodiments of the present disclosure will become apparent when studying the appended claims and the following description. The skilled person realize that different features of the example embodiments may be combined to create embodiments other than those described in the following, without departing from the scope of the present disclosure.

### BRIEF DESCRIPTION OF DRAWINGS

With reference to the appended drawings, below follows a more detailed description of embodiments of the disclosure cited as examples.

In the drawings:

FIG. 1a shows a so-called belted absorbent article comprising a waist belt according to the disclosure from the side that is intended to face towards the user when in use, in which the waist belt is joined to chassis of the absorbent article.

FIG. 1b shows a so-called belted absorbent article comprising a waist belt according to the disclosure from the side that is intended to face towards the user when in use, in which the waist belt is provided separate from the chassis of the absorbent article.

FIG. 2a shows an enlarged view of parts of the waist belt in FIG. 1, in which a first belt end section is provided with a fastening component and a second belt end section is provided with a mating fastening component.

FIG. 2b shows an enlarged view of the first belt end section in FIG. 2a, wherein said second transverse belt end section comprises a plurality of mating fastening component arranged spaced apart on the outer surface of said second transverse belt end section.

FIGS. 3a and 3b illustrate various embodiments of first and/or second connectors;

FIGS. 4a and 4b illustrate various embodiments of first and/or second connectors;

FIGS. 5a to 5d illustrate various embodiments of first and/or second connectors;

FIGS. 6a to 6c illustrate various embodiments of first and/or second connectors;

FIGS. 7a to 7c illustrate various embodiments of waist belt end sections comprising connectors;

FIG. 8 shows a so-called extension belt for a belted absorbent article according to the disclosure;

FIGS. 9a and 9b illustrate further embodiments of first and/or second connectors.

### DETAILED DESCRIPTION OF EXAMPLE EMBODIMENTS

Various aspects of the disclosure will now be described more fully hereinafter with reference to the accompanying drawings, in which exemplary embodiments of the disclosure are shown. The example embodiments of the disclosure may, however, be embodied in many different forms and should not be construed as limited to the embodiment set forth herein; rather, these embodiments are provided for thoroughness and completeness. Like reference character refer to like elements throughout the description.

The disclosure relates on the one hand to a disposable article of the belt type, a so-called belt article, the article comprising a transverse belt and a chassis part. Belt articles are characterised in that they comprise a transverse belt connected to either the front or rear end part of the article's chassis part. When applying such a belt article, the belt is fixed in a first stage around the user's waist. The chassis part of the belt article is hanging loosely from the belt in this case. The chassis part is then led between the user's legs and attached to the belt, wherein the belt comprises fixing surfaces intended to stick fast to the fixing member arranged on the chassis part by its free transverse edge.

The disclosure mainly refers to disposable absorbent articles, which means articles that are not intended to be laundered or otherwise restored or reused as absorbent articles after use.

The disclosure also relates to a disposable article intended to be fixed to a separate belt that is applied around the waist of a user. The disposable article has fastening components or fixing members at all its corners that are intended to be connected detachably to fastening surfaces or fixing surfaces arranged on the surface of the belt oriented away from the user.

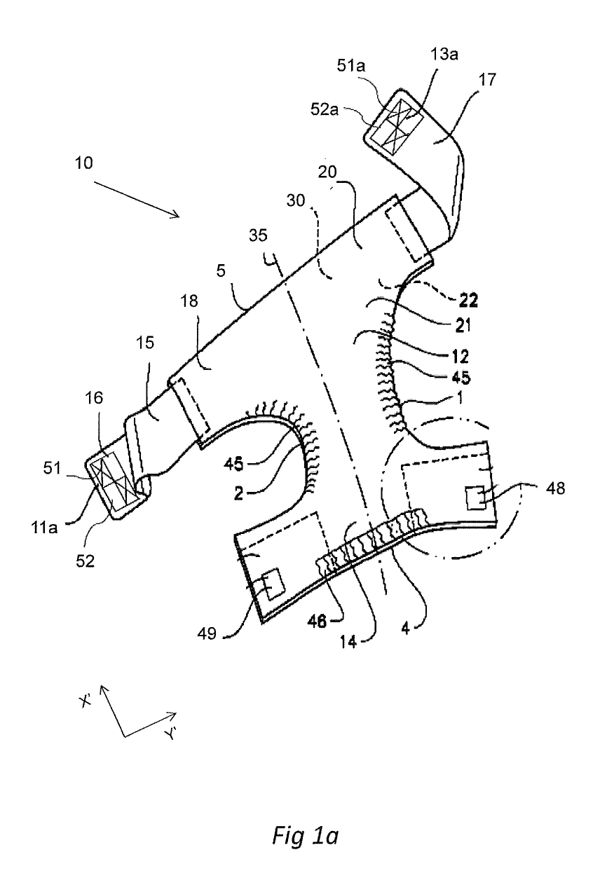

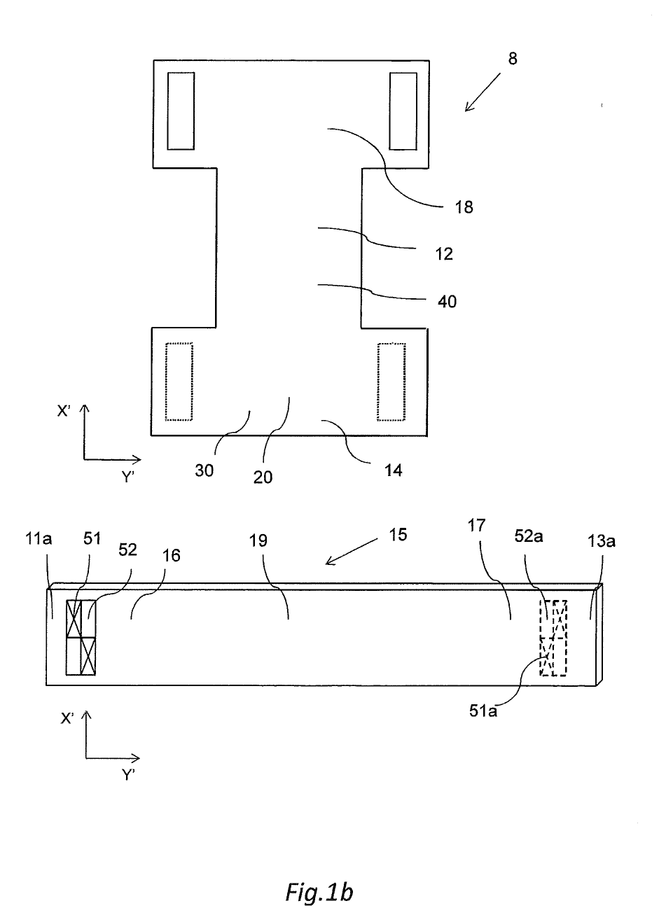

FIG. 1a shows substantial components of a belted article 10 according to the disclosure, wherein the belted article 10 is shown from the side which is intended to face towards the user when in use.

The belt article comprises a chassis and a waist belt. Further, the belt article 10 has a longitudinal direction X and a transverse direction Y and comprises a waist belt 15 and a chassis part 12 comprising a first covering layer 20. In other words, the belt article has a length extension in the longitudinal direction X′ and width extension in said transverse direction Y′.

The chassis, which includes the first covering layer 20, has a first surface 21 intended to face towards the user and a second surface 22 intended to face away from the user in use.

Typically, although not strictly required, the waist belt is partly or entirely elastic. Thus, hereinafter, the waist belt may be referred to as the waist elastic belt, the elastic belt or simply as the belt. The elastic belt 15 of the belt article 10 is intended to encircle a user's waist. As mentioned above, the elastic belt has an inner surface facing the wearer during use and an outer surface facing away from the wearer during use, wherein said belt comprising a first belt end section 16 and a second belt end section 17. In the belt article shown in FIG. 1a the first belt end section 16 is essentially a first belt half and the second belt end section 17 essentially a second belt half. Thus, in this example embodiment, the belt 15 has a first belt half 16 and a second belt half 17. The belt halves (first belt end section 16 and second belt end section 17) extend in the transverse direction Y′ of the belt article. More specifically, the chassis 12 has first and second end portions 18, 14 and a central portion 12a extending therebetween. The waist belt 15 is attached to the chassis 12 so that said first belt end section 16 and second belt end section 17 extends on each side of the first end portion of the chassis for securing to each other around a wearer of the article. To this end, as will be further described hereinafter, the first belt end section 16 has a fastening component and the second belt end section 17 has a mating fastening component for securing the belt end section to each other around the wearer of the article.

In this example embodiment, as shown in FIG. 1a, the first end portion is a rear end part and the second end portion is a front end part. Thus, the belt halves 16, 17 are connected to the chassis 12 in the rear end part 18, wherein the first belt half 16 is joined to the longitudinal edge 2 and the second belt half 17 is joined to the opposing longitudinal edge 1.

As briefly shown in the FIGS. 1a-1b, the first belt end section 16 is provided with a fastening component 11a and the second belt end section 17 is provided with a mating fastening component 13a. Further, each one of the fastening component 11a and the mating fastening component 13a comprises a first mechanical connector structure 51, 51a and a second mechanical connector structure 52, 52a. According to the example embodiments of the disclosure, each mechanical connector structure being capable of forming a mechanical interconnection with said other mechanical connector structure, and being incapable of forming a mechanical interconnection with an identical structure. Hereby, the fastening component 11a and the mating fastening component 13a are mechanically connectable via said first mechanical connector structure and said second mechanical connector structure to form an interconnection between said first belt end section and said second belt end section for securing to each other around a wearer of the absorbent article.

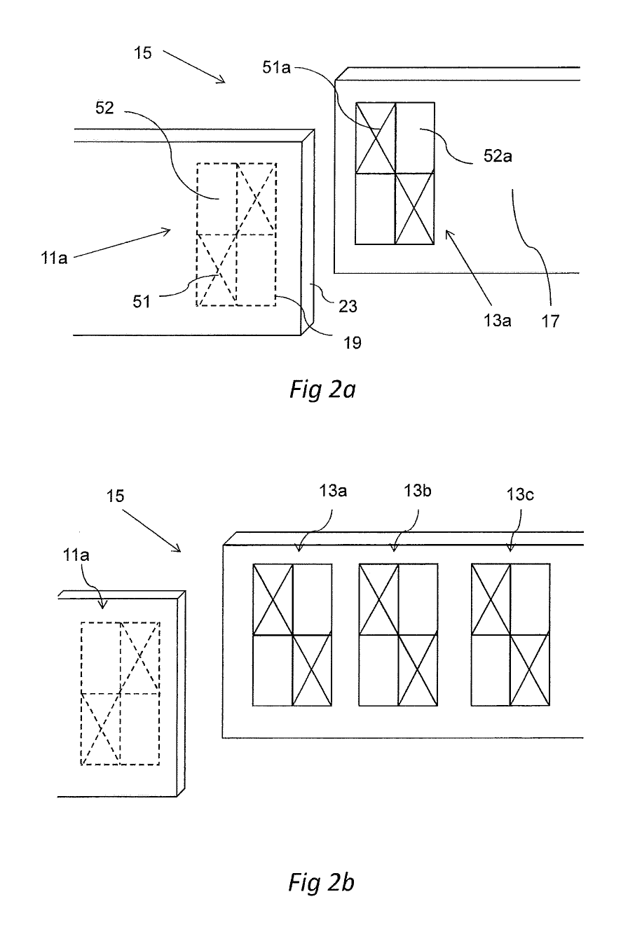

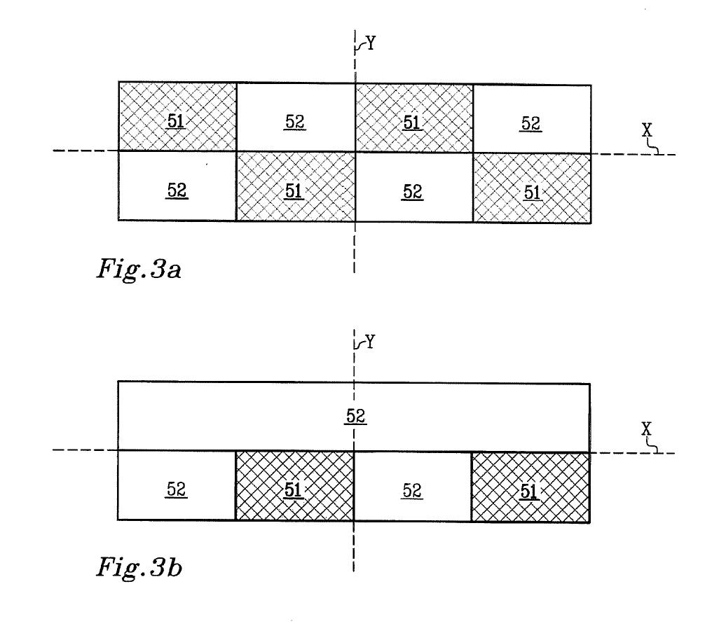

The fastening component and the mating fastening component, will described in particular with respect to FIGS. 2, 3a-9b hereinafter. It should be readily appreciated that the features, patterns, functions, examples and details of the fastening component as described in relation to the FIGS. 2, 3a-9b may likewise be incorporated into a mating fastening component of the waist belt, unless explicit stated herein. In addition, the example embodiments as described in FIGS. 2, 3a-9b is possible to be incorporated into a waist belt and absorbent article as described in relation to the FIGS. 1a and 1b, as mentioned above.

Turning again to FIGS. 1a-1b, the front end part, i.e. second end portion 14 of the chassis includes a fastening device 48, 49 for securing the second end portion of the chassis to the belt.

It is to be noted that the name belt half, or belt end section, does not mean that the two belt halves, or belt end sections, must be of the same length. In alternative embodiments, one belt half can be longer than the other. It is important, however, that the combined length of the two belt parts reaches around a user's waist. In an alternative aspect of the disclosure, the two belt halves 16, 17 of the belt article 10 can consist of only one piece of material, which piece of material extends in the transverse direction Y′ across the entire rear end part 18 and past the respective longitudinal edges 1, 2. The belt 15 can, in accordance with other alternative aspects of the disclosure, be inelastic or partly elastic. Partly elastic belt 15 means that certain parts of the length of the belt 15 have elastic properties, while certain other parts of the length of the belt do not have elastic properties.

Typically, although not strictly necessary, the belt article 10 comprises a first covering layer 20, wherein the covering layer comprises a first surface 21, intended to face towards the user when the belt article 10 is used, and an opposing second surface 22. The first covering layer 20 of the belt article 10 preferably has an hourglass shape, but other shapes such as a rectangular shape, for example, may also occur.

The first covering layer 20 can consist of any material suitable for the purpose. The most common breathable covering materials for disposable articles are nonwoven textile materials, so-called nonwoven materials manufactured according to various methods. Not so common but also occurring is the use of covering layers 20 of woven material or net material. For example, a nonwoven material suitable as a covering layer 20 can be manufactured from synthetic fibres such as polyester or polypropylene, of natural fibres such as cotton fibres. A mix of synthetic and natural fibres also occurs. The manufacture of a nonwoven material consists of the two main stages web formation and web bonding. Web formation means that a web is created of as even a surface weight as possible, wherein the web can be created by carding of natural or manmade staple fibres. The spunlaid technique is another web-forming technique, in which polymer material is extruded to thin continuous fibre threads on a continuous conveyor. The extruded thin fibre threads are randomised following extrusion in that they land on the continuous conveyor in a random manner. Web formation can take place in several different ways. A commonly occurring method is so-called thermal bonding, wherein a bonding pattern is melted into the fibre web in a roller nip where at least one of the hot rollers has a pattern. For web formation by means of thermal bonding to work, at least a certain portion of the fibres in the web must consist of synthetic fusible fibres. Occurring bonding patterns are dots of varying geometry, small rectangular or circular surfaces, floral patterns or the like. What is common to the methods described above, and also to other methods, is that not all the surface of the covering layer 20 is bonded, but between the bonding areas the covering layer has open areas comprising fibre threads that are fixed at one or both of their ends to the adjacent bonded areas.

Laminate consisting of two or more of the aforementioned possible covering materials also occur, such as a covering consisting of different materials in different parts of the surface. It also occurs that the covering layer 20 consists of an entirely or partially elastic material to give the belt article 10 a better fit when in use. The covering layer 20 may also have a fastening device (e.g. a first and a second fixing member 48, 49) arranged at the respective longitudinal edge 1, 2 in the front end part 14 of the covering layer 20, i.e. connected to the two front corners of the covering layer 20. The fixing members 48, 49 are arranged on the side of the covering layer 20 that is intended to face towards the user when the belt article 10 is used. The fastening device 48, 49 may typically comprise hook elements that are intended, on application of the belt article 10 to a user, to be connected detachably to loops arranged on the side of the belt 15 that is intended to face away from the user.

In other words, said second end portion 14 of the chassis typically includes a fastening device 48, 49 for securing the second end portion 14 of the chassis to the waist belt 15.

In alternative aspects of the disclosure, the fastening device 48, 49 can consist of separate fastening tabs that are connected permanently to the front end part 14 of the belt article 10, wherein the fastening tabs extend outside the edges of the belt article 10. The hook elements are arranged in this case on the fastening tabs.

When a belt article 10 is to be applied to a user, the belt 15 is first fixed around the user's waist. The front end part 14 of the belt article 10 that hangs loosely is then led in between the user's legs, following which the fastening device 48, 49 is fixed to the belt 15 on the stomach on the side of the belt 15 oriented away from the user. The hook elements of the fastening device are fixed in this case to loops arranged on the surface of the belt 15 oriented away from the user. A belt article is also conceivable in which the belt is connected to the front end part of the belt article. Such an article is applied to the user in the reverse manner, i.e. after the belt has been fixed around the user's waist, the loosely hanging rear end part is led in between the user's legs and fixed to the belt at the back on the side of the belt oriented away from the user.

To improve the fit of the belt article 10, the longitudinal edges 1, 2 of the first covering layer 20 can be provided with leg elastic 45 arranged substantially in the longitudinal direction X of the article. The task of the leg elastics 45 includes improving the fit of the article, and making the belt article 10 more like textile multiple-use briefs/pants. The respective leg elastic 45 can consist of one or more elastic threads that in the extended state have been joined to the covering layer 20 by gluing, ultrasonic welding or the like. Alternatively, the respective leg elastic 45 can consist of elastic ribbon material of foamed material, for example. The respective leg elastic 45 is preferably arranged on the side of the first covering layer 20 that is intended to face away from the user when in use.

The rear or front end part 18, 14 of the belt article 10 can also be provided with so-called waist elastic 46 in the form of elastic elements arranged along the second transverse edge 5 or first transverse edge 4 of the belt article 10 to give the belt article 10 a soft, flexible enclosure of the user's waist. In FIG. 1, only the front end part 14 of the belt article 10 is provided with waist elastic 46. The waist elastic 46 consists of a thin strip of elastic foam material that is attached by glue to the side of the first covering layer 20 that is intended to face away from the user. The waist elastic 46 is applied in a stretched state to achieve a holding-together force that stretches the belt article 10 around the user's waist. When the fastening device 48, 49 are fastened to the belt 15, it is suitable that the waist elastic 46 is tensioned in the transverse direction Y of the belt article 10 so that the front end part 14 has a smooth configuration over the user's stomach on use.

According to another aspect of the disclosure, the belt article 10 comprises a second covering layer 30 arranged on the side of the first covering layer 20 that is intended to face away from the user when in use. The second covering layer 30 has the same extension in the X/Y plane as the first covering layer 20. The second covering layer 30 is preferably substantially liquid-impermeable, but other types of covering layer can also exist. The second covering layer 30 can consist of a range of different materials, but most frequently it consists of a thin, preferably liquid-impermeable plastic film, but it is also possible to use other types of substantially liquid-impermeable material such as nonwoven material, for example, that has been made substantially liquid-impermeable through plastic coating, for example, a liquid-impermeable foam layer, liquid-impermeable glue or the like. The second covering layer 30 can alternatively consist of a liquid-impermeable vapour-permeable material, a so-called breathable material. Furthermore, laminates comprising at least one liquid-impermeable and preferably breathable layer arranged against the second surface 22 of the first covering layer 20 also exist as a second covering layer 30. These laminates usually consist of a liquid-impermeable material acting as a barrier against soaking through and a more textile-like material arranged on the side of the belt article 10 that is oriented away from the user on use. The outside of the belt article 10 is thereby more garment-like on use. The textile-like layer of the laminate normally consists of a nonwoven layer, but other textile or textile-like materials also exist. The first covering layer 20 and the second liquid-impermeable covering layer 30 can be joined to one another in several different ways. Examples of joining methods are gluing, thermal fusing, ultrasonic welding or the like. For belt articles 10 comprising a first covering layer 20 and a substantially liquid-impermeable second covering layer 30, it is suitable for the leg elastic 45 and the waist elastic 46 described above to be arranged between the two covering layers 20, 30. The first covering layer 20 can, in a belt article 10 comprising a liquid-impermeable second covering layer 30, have a low absorption capacity wherein smaller bodily secretions such as occasional drops of urine, for example, initially secreted menstruation fluid or similar.

According to an example embodiment of the disclosure, the belt article 10 has an absorption body 40 arranged between the first covering layer 20 and the second substantially liquid-impermeable covering layer 30. The first covering layer 20 of a belt article according to this aspect must be liquid-permeable. The belt article 10 in accordance with this aspect relates to a diaper intended to be used by an incontinent adult person or by a child who has not yet become continent. The absorption body 40 has substantially the same profile, but a smaller surface, than the first and second covering layers 20, 30. The two covering layers 20, 30 thus extend outside the edges of the absorption body 40 along the entire circumference of the absorption body 40. The absorption body 40 has, exactly like the covering layers 20, 30, a front and a rear end part and a narrower crotch part located between the end parts. Upon use of a belt article comprising an absorption body 40, the front part of the crotch part and the front end part principally act like a receiving area for urine, while the rear part of the crotch part and the rear end part act mainly as a receiving area for faeces. The liquid-permeable first covering layer 20 and the substantially liquid-impermeable second covering layer 30 are connected to one another outside the absorption body 40 along its entire circumference. The absorption body 40 can be constructed from one or more layers of cellulose fluff pulp. The cellulose fluff pulp can be mixed with fibres or particles of a highly absorbent polymer material, so-called super absorbents, of the type that chemically binds large quantities of fluid on absorption with the formation of a fluid-holding gel. The absorption body 40 can also comprise highly absorbent polymer material arranged in a layer inside the absorption body or connected to the surface or surfaces of the absorption body. The absorption body 40 can further include further components for improving the properties of the absorption body 40. Examples of such components are binding fibres, various types of fluid-dispersing layers or fibres, dimensionally stabilising components, reinforcing fibres or the like.

FIG. 1b shows another type of disposable article 8 in accordance with the disclosure, wherein the disposable article 8 is intended to be connected detachably to a separate belt that is applied around a user's waist. Thus, said article 8 here comprises a chassis 12 and a waist belt 15 according to any one of the features, example, function or aspects as mentioned above, wherein said waist belt 15 is provided separate from the chassis 12. Typically, an intermediate belt section 19 is arranged between said first belt end section 16 and said second end belt section 17 to form a complete waist belt intended for use with an absorbent article.

As mentioned previously herein, the disposable article 8 has a chassis comprising a first covering layer 20 constructed in the same way as the first covering layer 20 of the belt article 10. The disposable article 8 can comprise a second covering layer 30 and an absorption body 40 in accordance with other aspects of the disclosure. The first covering layer 20, second covering layer 30 and the absorption body 40 are constructed and arranged here in the same way as they are constructed and arranged for the belt article 10 described above. The disposable article 8 has an hourglass shape, but according to alternative aspects of the disclosure it may have any shape, such as a rectangular shape, for example. The disposable article 8 is substantially designed symmetrically and can therefore be turned with the rear end part 18 to the front on the user without any problem; the term back or front end part 18, 14 is intended more to distinguish the end parts in this description.

The disposable article 8 can incorporate a waist belt 15 according to any one of the example embodiments described herein.

FIG. 2a depicts an enlarged view of a section of the waist belt 15 in FIGS. 1a and 1b. As mentioned above, said first belt end section 16 of the waist belt is intended to be interconnected to second belt end section 17 by the fastening component and the mating fastening component for securing to each other around a wearer of the absorbent article. The first belt end section is provided with the fastening component 11a and the second belt end section is provided with the mating fastening component 13a, wherein each one of the fastening component 11a and the mating fastening component 13a comprises said first mechanical connector structure 51, 51a and said second mechanical connector structure 52, 52a. That is, the fastening component 11a comprises the first mechanical connector structure 51 and the second mechanical connector structure 52, whilst the mating fastening component 13a comprises the first mechanical connector structure 51a and the second mechanical connector structure 52a. Said first and second mechanical connector structures being two corresponding structures. Each mechanical connector structure being capable of forming a mechanical interconnection with said other mechanical connector structure, and being incapable of forming a mechanical interconnection with an identical structure. To this end, the first mechanical connector structure is different than the second mechanical connector structure. As an example, the first mechanical connector structure is hook connector structure and the second mechanical connector structure is a loop connector structure.

Thus, the first mechanical connector structure 51 of said fastening component 11a is different than said second mechanical connector structure 52a of said mating fastening component 13a and the second mechanical connector structure 52 of said fastening component 11a is different than said first mechanical connector structure 51a of said mating fastening component 13a. As such, said first mechanical connector structure of said fastening component is adapted to interconnect with said second mechanical connector structure of said mating fastening component, and said second mechanical connector structure of said fastening component is adapted to interconnect with said first mechanical connector structure of said mating fastening component.

Hereby, said fastening component 11a and said mating fastening component 13a are mechanically connectable via said first mechanical connector structure 51, 51a and said second mechanical connector structure 52, 52a to form an interconnection between said first belt end section and said second belt end section for securing to each other around a wearer of the absorbent article. In this context, an interconnection refers to a configuration when said first belt end section is joined to said second belt end section.

Accordingly, the fastening component 11a is mechanically connectable to the mating fastening component 13a provided said mating fastening component comprises the first and second mechanical connector structures.

To this end, said fastening component 11a is adapted to interconnect with said mating fastening component 13a. Analogously, said mating fastening component 13a is adapted to interconnect with said fastening component 11a.

Optional, although not strictly required, the location of the corresponding mechanical connector structures on the respective fastening components, and the relative orientation thereof, are such that the corresponding connector structures may entirely meet to form an interconnection. Thus, the size and orientation of the fastening component on the first belt end section, as seen in the direction x and the direction y, typically corresponds to the size and orientation of the mating fastening component on the second belt end section, as seen in the direction x and direction y.

Typically, said first mechanical connector structure 51 and the second mechanical connector structure 52 of said fastening component is arranged in a first pattern and the first mechanical connector structure 51a and the second mechanical connector structure 52a of said mating fastening component is arranged in a second pattern. In addition, said first pattern and said second pattern are formed so that at least a portion of the first mechanical connector structure 51 of said fastening component mirrors at least a portion of the second mechanical connector structure 52a of said mating fastening component and at least a portion of the second mechanical connector structure 52 of said fastening component mirrors at least a portion of the first mechanical connector structure 51a of said mating fastening component.

As an example, FIG. 2a illustrates an example embodiment of the disclosure wherein the first mechanical connector structure 51 and the second mechanical connector structure 52 of said fastening component 11a is arranged in a first pattern and the first mechanical connector structure 51a and the second mechanical connector structure 52a of said mating fastening component 13a is arranged in a second pattern. As shown, said first pattern is a mirror image of said second pattern.

In other words, the arrangement of the first mechanical connector structure 51 and the second mechanical connector structure 52 of said fastening component is a mirror image of the arrangement of the first mechanical connector structure 51a and the second mechanical connector structure 52a of said mating fastening component.

In this manner, the first mechanical connector structure 51 and the second mechanical connector structure 52 of the fastening component and the first mechanical connector structure 51a and the second mechanical connector structure 52a of the mating fastening component are capable of interconnecting over the entire area of the two different connector structures, thereby increasing the interconnection between waist belt end sections to a maximum.

Due to the characteristic of the mechanical connector structures, the fastening component of the first second belt half is intended to be connected detachably to the mating fastening component 13a of the second belt half 17 when the belt 15 is applied around the waist of the user. The outer edge 19 of the fastening component 11a is here arranged a small distance from the free end 23 of the belt half 16. Alternatively, it may also occur that the outer edge 19 of the fastening component 11a coincides with the free end 23 of the belt half 16. It may also occur that the fastening component 11a is as wide as the belt half 16 is wide.

The fastening component and the mating fastening components can be bonded to the belt end sections in a variety of ways known in the art, such as ultrasonic welding, adhesive or a combination thereof. Typically, as illustrated in FIG. 2a, the fastening component 11a is arranged on the inner surface of the first belt end section 16 and the mating fastening component 13a is arranged on the outer surface of the second belt end section.

The fastening components 11a and the mating fastening components 13a are mechanical connectors. Mechanical connectors have the advantage of providing a secure connection between themselves, whilst not being prone to unwanted connection to other materials. Hence, use of mechanical connectors facilitates the general fastening of belt sections of the articles to maintain the waist belt and the article on the wearer during use of the article. The fastening components and the mating fastening components are thus particularly suitable for mechanically interconnecting belt end sections to each other.

The fastening components and the mating fastening components are thus configured to allow the waist belt to be detachable connected, which also provides the possibility of correcting the position of the waist belt if a user has unintentionally performed an unsuitable interconnection between said end belt sections of the article.

Generally speaking, the fastening component and the mating fastening component may be configured to connect at a variety of angles. While the drawings generally illustrate an essentially identical fastening component meeting with a corresponding mating fastening component, it is to be noted that the fastening component and the mating fastening component must necessarily not be identical in terms of pattern, shape and/or geometry, but that the width, length, pattern and/or shape of the fastening component may be different to the width, length, pattern and/or shape of the mating fastening component.

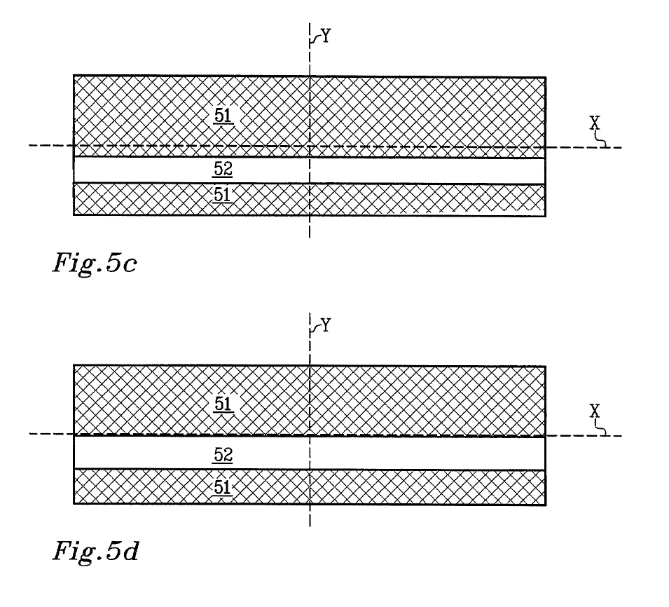

In this context, it is to be noted that a fastening component and/or a mating fastening component as described in relation to FIG. 5a herein is particularly useful for a waist belt as described above in relation to FIG. 1a, 1b and FIG. 2b. However, other possible pattern and shapes are conceivable as described hereinafter.

FIG. 2b illustrates another example embodiment, wherein said second transverse belt end section 17 comprises a plurality of mating fastening component 13a, 13b and 13c arranged spaced apart on the outer surface of said second transverse belt end section. Further, each mating fastening component comprises first mechanical connector structure and second mechanical connector structure, so that said fastening component and any one of the plurality of said mating fastening components are mechanically connectable via said first mechanical connectors structure and said second mechanical connector structures to form an interconnection between said first belt end section and said second belt end section for securing to each other around a wearer of the absorbent article.

Accordingly, the disposable article in the example embodiments of the disclosure, as particularly described in relation to FIG. 1a, 2a or 2b, comprises the chassis and the waist belt, said chassis having first and second end portions and a central portion extending therebetween, and including a first covering layer 20 having a first surface 21 intended to face towards the user and a second surface 22 intended to face away from the user in use. Further, said waist belt is attached to the chassis so that said first transverse belt end section and second transverse belt end section extend on each side of the first end portion of the chassis for securing to each other around a wearer of the article, and wherein said second end portion of the chassis includes a fastening device 48, 49 for securing the second end portion of the chassis to the belt.

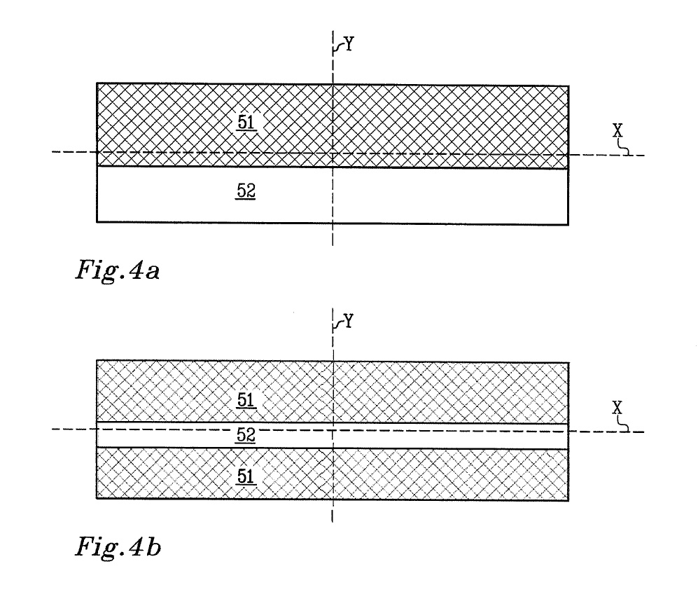

In the example embodiments herein, as shown in e.g. FIG. 1a-1b, 2a-2b and FIGS. 3a-9b, it is envisaged that the fastening component and the mating fastening components both comprise first mechanical connector structure, e.g. 51, 51a and second mechanical connector structure, e.g. 52, 52a, respectively.

In certain variants, the first and second mechanical connector structures 51, 52 of a fastening component may be arranged to form a pattern over a surface of the belt end section. The example embodiments described in relation to FIGS. 3a-3b, 4a-4b, 5a-5c are all example embodiments where the first and second mechanical connector structures 51, 52 are arranged so as to form organised patterns.

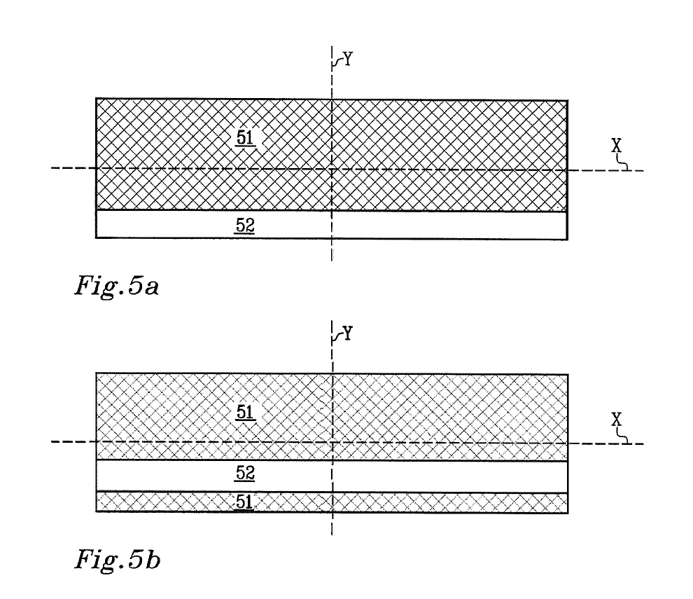

In certain variants, the pattern may be adapted such that a portion of the first belt end section is mechanically connectable to a portion of the second end belt section via the fastening component and the mating fastening component. At least the variants described in FIGS. 3a-3b and 4a-4b are adapted to such use. It will be noted, that for being mechanically connectable to the end belt section via the fastening component and the mating fastening component, there may still be requirements as to the orientation of the fastening component and the mating fastening component in order to achieve such a connection.

In certain variants, the pattern may be adapted such that the first end belt section is mechanically connectable to the second end belt section via the fastening component and the mating fastening component, regardless of how the respective end belt sections are oriented in relation to each other. In other words, the connecting system is fool-proof in that all conceivable combinations when trying to set identical fastening component and mating fastening component together will be successful. FIGS. 3a-3b illustrate a such a variant of a fastening component and a mating fastening component.

In FIGS. 3a-3b, FIGS. 4a-4b, and FIGS. 5a-5b, various fastening components are illustrated with reference to a longitudinal axis X and a transversal axis Y. It is to be noted, that although the figures illustrate the fastening component only, the axes are essential defined in relation to the longitudinal direction x′ and the transverse direction y′ of the article.

It should be readily conceivable that the description in relation to the figures mentioned above is likewise applicable to various mating fastening components of the example embodiments.

Typically, the fastening component (and mating fastening component) each defines a longitudinal central axis X extending in parallel to the length the fastening components, and centrally in relation to the width of the fastening components. Similarly, the fastening component (and mating fastening component) each defines a longitudinal transverse axis Y extending in parallel to the width of the fastening components, and centrally in relation to the length of the fastening components. The same description may also apply to the extension and direction of the belt end sections.

FIG. 3a illustrates a variant of a fastening component 11a where the pattern formed by the first and the second mechanical connector structures 51, 52 is asymmetrical with respect to said longitudinal central axis X, and with respect to the transversal central axis Y.

Moreover, the pattern formed by the first and second connector structures 51, 52 is such that each portion of the first structure 51 on one side of the longitudinal central axis X, mirrors a portion of the second structure 52 on the other side of the longitudinal central axis X. Similarly, each portion of the first structure 51 on one side of the longitudinal transversal axis Y, mirrors a portion of the second structure 52 on the other side of the transversal central axis Y.

This results in a “chess-board” patterned fastening component 11a as illustrated in FIG. 3a.

The arrangement with the mirroring first and second connector structures 51, 52 results in a fastening component which, when applied on both the end belt sections results in a fool-proof interconnection of said belt end sections. No matter in which orientation the connectors are situated when they meet each other, each portion of the first mechanical connector structure 51 will always meet a portion of the second mechanical connector structure 52, resulting in an interconnection between said portions. Hence, the likelihood of an interconnection being created, if two such fastening components (fastening component and mating fastening component) randomly put together, is 100%.

FIG. 3b illustrates another variant of a fastening component 11a, where the pattern formed by the first and the second connector structures 51, 52 is asymmetrical with respect to said longitudinal central axis X, and with respect to the transversal central axis Y.

In this case, on one side of the longitudinal central axis X only the second connector structures 52 appears. On the other side of the longitudinal central axis X, there is a pattern comprising portions of the first connector structure 51, and of the second connector structure 52, arranged in an alternating manner and mirroring each other as seen over the transversal axis Y.

In the illustrated arrangement, each portion of the first structure 51 on one side of the longitudinal central axis X, mirrors a portion of the second structure 52 on the other side of the longitudinal central axis X. Similarly, each portion of the first structure 51 on one side of the longitudinal transversal axis Y, mirrors a portion of the second structure 52 on the other side of the transversal central axis Y. Accordingly, also the fastening component illustrated in

FIG. 3b results in a fool-proof interconnection between fastening components provided with such connectors on both of their end belt sections. No matter in which orientation the fastening component and the mating fastening component are when they meet each other, some portion of the first structure 51 of the fastening component will always meet a portion of the second structure 52 of the mating fastening component, and result in an interconnection. Hence, the likelihood of an interconnection being created, if two such fastening components (fastening component and mating fastening component) are randomly put together, is 100%.

In the embodiment illustrated in FIG. 3b, all portions of the first mechanical connector structure 51 may not meet a portion of the second mechanical connector structure 52 in all possible relative orientations of the fastening component and the mating fastening component. This is, however, not required for ensuring connection between the fastening component and the mating fastening component. Accordingly, the arrangement illustrated in FIG. 3b might be perceived as a simplified variant of the arrangement of FIG. 3a.

Indeed, in order to provide at least one portion of the first structure 51, located on one side of the longitudinal central axis X and on one side of the transversal central axis Y; mirroring a portion of the second structure 52 on the other side of the longitudinal central axis X and another portion of the second structure 52 on the other side of the transversal central axis Y, so as to render the connection between two such fastening component and mating fastening component fool-proof, it would be sufficient to provide a fastening component similar to the one illustrated in FIG. 3b, but comprising only one single portion of the first structure 51.

FIG. 4a illustrates an example of a fastening component where a first mechanical connector structure 51 and a second mechanical connector structure 52 are asymmetrically arranged with respect to the longitudinal axes X, but symmetrically arranged with respect to the transversal axes Y. Analogously, FIG. 4a may illustrate an example of a mating fastening component where a first mechanical connector structure 51a and a second mechanical connector structure 52b are asymmetrically arranged with respect to the longitudinal axes X, but symmetrically arranged with respect to the transversal axes Y.

Although such arrangements will generally provide less versatility as to the interconnection between the fastening component and the mating fastening component, they may nevertheless contribute to an ease of manufacturing.

The example embodiment of a fastening component in FIG. 4a comprises a portion of the first connector structure 51, which extends on one side of the longitudinal axis X, and slightly over said axis X. A portion of the second connector structure 52 is hence arranged at a distance from the longitudinal axis X, and extending further away from the longitudinal axis X. The portions of the first and the second connector structure 51, 52 both extend in a band-shaped manner along the longitudinal axis X.

A fastening component as illustrated in FIG. 4a may be mechanically connected to an identical mating fastening component, if the identical fastening component and mating fastening component are positioned in relation to each other such that the first connector structure 51 of the fastening component meets the second connector structure 52a of the mating fastening component. Hence, configurations comprising identical such fastening components on both end belt sections would, if randomly oriented, display a likelihood of becoming interconnected of 50%

However, the embodiment of a fastening component in FIG. 4a may be used together with another mating fastening component, e.g. as illustrated in FIG. 4b, to facilitate interconnections of the configuration. The fastening component as illustrated in FIG. 4b comprises two portions of the first connector structure 51, extending along the longitudinal axis X. A portion of the second connector structure 52 is further arranged in between the two portions of the first connector structure 51.

The second connector structure 52 of the fastening component of FIG. 4b is arranged in a staggered relationship to the second connector structure 52a of the mating fastening component in FIG. 4a. Accordingly, a fastening component in accordance with FIG. 4b will be connectable to a mating fastening component in accordance with FIG. 4a, regardless of how the fastening component and the mating fastening component are oriented in relation to each other.

Accordingly, a configuration may be provided with a fastening component 11a on an end belt section, which is in accordance with FIG. 4a, and with a fastening component 13a on another end belt section, which is accordance with FIG. 4b. In this case, the likelihood of achieving an interconnection between a fastening component and a mating fastening component, when no concern is taken to the relative orientation of the components may be about 75%.

As understood by the examples of FIGS. 3a-3b, and FIGS. 4a-4b, various designs and example embodiments of the fastening components and the mating fastening components intended for interconnection with each other via first and second mechanical connectors provided on said fastening component and said mating fastening component may be provided such that the first and second mechanical connectors of the fastening component is mechanically connectable to the first and second mechanical connectors of the mating fastening component.

In this case, the first and second mechanical connectors of said fastening component and said mating fastening component may also be identical. Alternatively, the first and second connector of said fastening component and said mating fastening component may be different.

Another option is to provide said fastening component and said mating fastening component having first and second mechanical connectors, where the appearance of the first and/or second mechanical connector may differ between the fastening component of one end belt section and the mating fastening components of another end belt section.

For example, the fastening component may display a first pattern, whilst the mating fastening component may display a second pattern. In this case, the connection possibilities between the fastening component and mating fastening component might be different, whilst still providing a sufficient level of connection strength. Nevertheless, by selecting suitable patterns, and suitable variations between patterns, it may be ensured that a sufficient number of connection possibilities is obtained.

FIGS. 5a to 5d illustrate some variants of the fastening component and the mating fastening component that may be used for such embodiments. In these variants, a fastening component 11a is symmetrically arranged in view of the longitudinal and transversal axis X, Y. A first connector structure 51 extends over the entire area spanned by the fastening component 11a, apart from over a band shaped area extending along the transversal axis X, and which is occupied by a second connector structure 52. From FIGS. 5a-5d, it may be seen how the location of the band-shaped area of the second connector structure 52 varies between the variants of the figures, in that the distance between the band-shaped area of the second connector structure 52 and the transversal axis X varies. The area comprising the second connector structure 52 may be described as “wandering” over the area of the fastening component 11a.

To this end, as shown by the example embodiments, the fastening component and mating fastening component will always be connectable to each other, regardless of the orientation of said fastening component and said mating fastening component. However, if the fastening component is to be connected to another, identical mating fastening component, there is a risk that no connection will occur, should two identical components meet when identically orientated.

In the embodiments described in relation to FIGS. 5a-5d, the different patterns of the fastening component and mating fastening component are obtained by varying a selected pattern in a controlled manner. Such variations may result e.g. if using a continuous connector material displaying a repeated pattern for forming the fastening component and mating fastening component, and where the pattern repetition is not evenly divisible with the selected component length. In this case, for each fastening component and mating fastening component cut from the connector material, the pattern will “wander” a distance corresponding to the mismatch between the pattern repetition length and the component length.

Other embodiments are possible where the fastening component and mating fastening component are provided with randomly selected or varied patterns.

To provide the fastening component and the mating fastening component 11a, 13a on the end belt sections numerous alternatives are possible.

For example, the first connector structure 51 may be provided by a first connector material 51′, and the other connector structure is provided by a second connector material 52′.

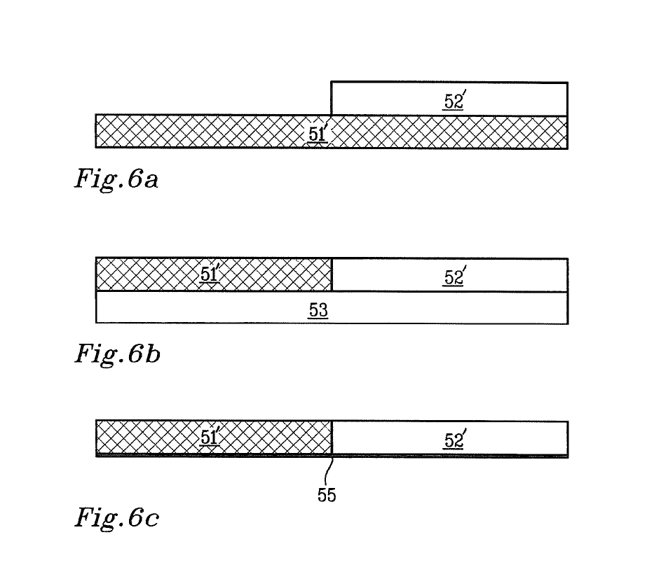

FIGS. 6a to 6c illustrate various variants of fastening components and mating fastening components, viewed in a cross-section along the axis Y.

FIG. 6a illustrates a variant wherein the second connector material 52′ is attached to the first connector material 51′. The first connector material 51′ may then be attached to the respective end belt section by adhesive, ultra-sonic welding or the like.

Using this variant, a variant of a fastening component or mating fastening component such as the one illustrated in FIG. 3a or 3b may be manufactured by attachment of intermittent pieces of second connector material 52′ onto a continuous piece of first connector material 51′.

In another variant, a first connector material 51′ may be provided as a continuous piece of material. A second connector material 52′ may be provided as a piece of material in which through holes are formed. The second connector material 52′ may then be applied over the first connector material 51′, such that the first connector structures 51 of the first connector material 51′ is accessible via the through holes in the second connector material 52′. The first connector material 51′ may then be attached to the respective end belt section.

For example, in accordance with the last mentioned variant, a continuous piece of second connector material 52′, in which through holes are cut out, may be laminated over a continuous piece of first connector material 51′, resulting in a continuous piece of connector material comprising first mechanical connector structures 51 and second mechanical connector structures 52, which may be used to form fastening components and mating fastening components 11a, 13a. The first connector material 51′ may in this case be a loop material, and the second connector material 52′ may be a hook material.

In the above-mentioned example, the through holes may be cut out randomly, so as to provide different fastening component or mating fastening component displaying random variations.

Alternatively, the through holes may be cut out in a selected pattern. A connector material comprising a selected pattern may be used for forming identical fastening component and mating fastening component (e.g. if the pattern repetition is evenly divisible with the length of the fastening component and mating fastening component), or different fastening component and mating fastening component (e.g. if the pattern repetition is not evenly divisible with the length of the fastening component and mating fastening component).

FIG. 6b illustrates a variant wherein the fastening component 11a comprises a carrier material 53, onto which said first and second connector material 51′, 52′ is attached. The carrier material 53 is typically attachable to the respective belt end section 16, 17.

The variants exemplified by FIGS. 6a and 6b may be formed directly on the end belt sections, after manufacture thereof.

FIG. 6c illustrates an embodiment of a fastening component, wherein the first mechanical connector structure 51 and the second mechanical connector structure 52 is provided by a single continuous connector material. Such a material may comprise a backing 55 from which the mechanical connectors structures 51, 52 extend.

In the examples of FIGS. 6a to 6c, the fastening component is illustrated as a unitary fastening component, forming a continuous piece of material which may be attached to the belt end section 16, 17. Naturally, other variants of unitary fastening components are conceivable.

However, a fastening component may also be formed by attachment of one or several separate material pieces of connector structure material intermittently arranged on the end belt section. In this case, the fastening component may be described as an intermittent fastening component. Analogously, a mating fastening component may also be formed by attachment of one or several separate material pieces of connector structure material intermittently arranged on the mating end belt section. In this case, the mating fastening component may be described as an intermittent mating fastening component.

Furthermore, according to some variants, each one of the end belt sections may comprise a set of fastening components bonded to said end belt section. The set of the fastening components typically comprise a plurality of fastening components arranged spaced apart as seen in the longitudinal direction. In addition, or alternatively, the plurality of fastening components may be arranged spaced apart as seen in the transverse direction.

Analogously, the second end belt section may comprise a set of mating fastening components bonded to said second end belt section. The set of the mating fastening components typically comprise a plurality of mating fastening components arranged spaced apart as seen in the longitudinal direction. In addition, or alternatively, the plurality of mating fastening components may be arranged spaced apart as seen in the transverse direction.

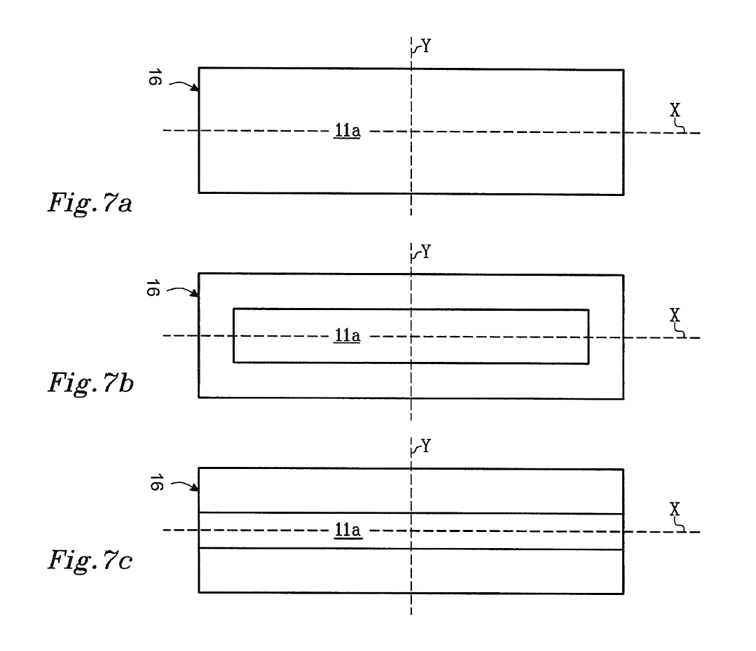

As illustrated by FIG. 7a, the fastening component 11a may be arranged so as to extend over essentially the entire end belt section as seen in the direction X. It should be noted that the FIGS. 7a-7c merely shows a cut-out view of the end belt section of the article so as to simplify the description and illustration of the various variants.

If the fastening component is a unitary fastening component, e.g. in accordance with the examples of FIGS. 6a to 6c, this means that the entire end belt section as seen in the direction X will be covered by the fastening component 11a.

Alternatively, the fastening component may extend over only a portion of the length L or width W of the end belt section.

FIG. 7b illustrates an arrangement where the fastening component 11a extends over only a portion of the length L and over only a portion of the width W of the end belt section. In this case, the fastening component 11a is centrally arranged in view of said width and length (and consequently in view of said longitudinal and transverse axis X, Y).

FIG. 7c illustrates an arrangement where the fastening component 11a extends over the entire length L, but only over a portion of the width W of the end belt section. The fastening component 11a is centrally arranged in view of said width and length (and consequently in view of said longitudinal and transverse axis X, Y).

Central arrangement of the fastening component on end belt section, may be used to ensure that the fastening component arranged on said end belt section and the mating fastening component arranged on said second end belt section will meet in a controlled manner. In this case, any desired asymmetry of the first and/or second surface structure in relation to the longitudinal and/or transversal axis is obtained by the arrangement of the surface structures in relation to the fastening component.

However, variants are conceivable wherein any desired asymmetry of the first and/or second surface structure in relation to the longitudinal and/or transversal axis is at least partly obtained by asymmetrical arrangement of the fastening component on the end belt section.

The attachment of the fastening component 11a to an end belt section may be performed directly or indirectly. Advantageously, the attachment may be an adhesive attachment, performed e.g. by gluing, or by the provision of the fastening component as an adhesive sticker. Alternatively, the attachment of the fastening component to an end belt section may be performed by ultra-sonic welding to form an ultra-sonic bond.

The fastening component 11a and the mating fastening component 13a may each have a height of less than 2 mm, preferably less than 1 mm, most preferred 0.6 mm. The height is to include the entire connector, including e.g. backing materials etc.

The shear force between the fastening component and the mating fastening component, when interconnected, reflects the strength of the interconnection. The fastening component and the mating fastening component should be selected such that, when connected, the interconnection is able to resist the forces involved when the article is to be used in its belted configuration.

It is understood, that in the various examples of the configurations, the fastening component and the mating fastening component may be combined with each other and/or with features described in relation to any one of the examples.

If desired, the pattern formed by the first mechanical connector structure and the second mechanical connector structure may be used as a visual indicia for indicating a correct fastening so as to form a comfortable and secure belted configuration during use of the article. Such an indicia may be useful in particular when the article and its fastening component and mating fastening component are adapted so as to provide a higher likelihood of interconnection.

The various fastening components and mating fastening components proposed herein are particularly useful for belted articles being designed to allow a user or caregiver to open and reclose the article as desired.

In view of the above disclosure explaining how to provide fastening component and mating fastening component for achieving an interconnection between end belt section, it will be understood that the article is provided with improved fastening ability in terms of secureness and ease of handling.

Different patterns formed by the first and second connector structures in order to provide a suitable fastening component may be designed, and such patterns may be combined in different manners. Although several of the patterns given in the illustrated examples display continuous rectangular areas comprising the first or the second mechanical connector structures, patterns may naturally be formed by other intermittent or continuous areas, having any desired shape, e.g. triangular, circular, half circular or the like.

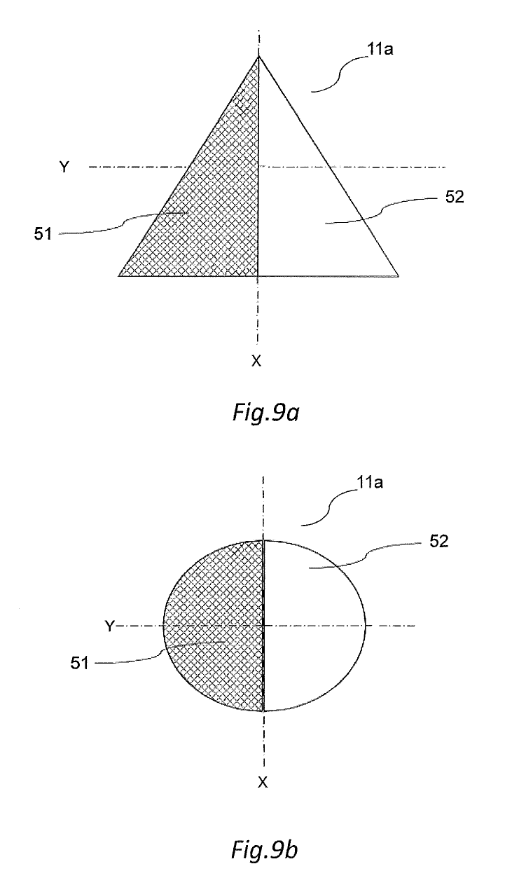

As an example, FIG. 9a illustrates one example embodiment of a fastening component in the shape of a triangle, in which the first and second mechanical connector structures 51, 52 are provided as right angled triangles, respectively.

FIG. 9b illustrates another example embodiment of a fastening component in the shape of a circle, in which the first and second mechanical connector structures 51, 52 are provided as half circles, respectively. It should be readily appreciated that the shapes illustrated in FIGS. 9a and 9b may also be used for the shape of the mating fastening component. Accordingly, it is to be noted that the fastening component and the mating fastening components, as well as the pattern of the mechanical connectors may be provided in several different forms and shapes depending on the design and function of the waist belt, belted article and the use thereof.

Typically, one of said first and second mechanical connector structures 51, 52 comprises hooks, and the other of the first and the second connector structures 51, 52 comprises loops.

It is also to be noted that the mechanical connectors are necessarily a hook material and a loop material, respectively. Thus, in all example embodiments of the disclosure, one of said first and second connector structures 51, 52 (51a, 52a) may comprise a first type of hooks, and the other of the first and the second connector structures 51, 52 (51a, 52a) may comprise a second type of hooks, said first type of hooks having a different hook characteristics than said second type of hooks.

Further, as mentioned above, any one of the first belt end section and the second belt end section may in some example embodiments comprise an elasticized region.

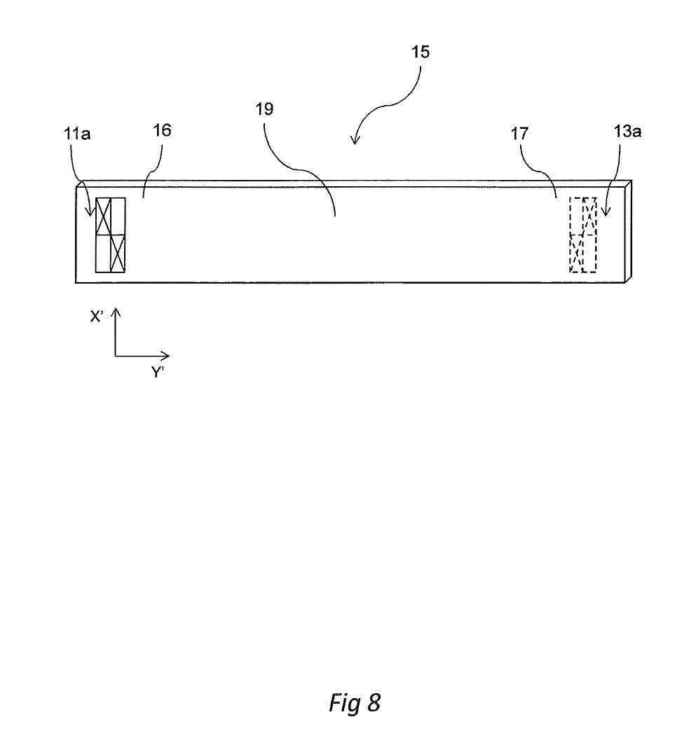

In addition, it is also possible that said waist belt is an extension belt product 15′ for an absorbent article, as shown in FIG. 8. As shown in FIG. 8, an intermediate belt section 19 is further arranged between said first belt end section and said second end belt section to form a waist belt extension intended for use with an absorbent article.

The term “nonwoven” is applied to a wide range of products which in term of their properties are located between the groups of paper and cardboard on the one hand and textiles on the other hand. As regards nonwovens a large number of extremely varied production processes are used, such as airlaid, wetlaid, spunlaced, spunbond, meltblown techniques etc. The fibres may be in the form of endless fibres or fibres prefabricated with an endless length, as synthetic fibres produced in situ or in the form of staple fibres. Alternatively, they may be made from natural fibres or from blends of synthetic fibres and natural fibres.

By “absorbent article” is meant an article that absorbs or is adapted to absorb bodily fluids, such as urine and/or blood.

The disclosure also covers all conceivable combinations of the described aspects, variants, alternatives and example embodiments of the disclosure.

Furthermore, the disclosure is not limited to the aforesaid aspects, but is naturally applicable to other aspects within the scope of the following claims.

Reference signs mentioned in the claims should not be seen as limiting the extent of the matter protected by the claims, and their sole function is to make claims easier to understand.

1-23. (canceled)

24. A disposable absorbent article comprising a chassis and a waist belt, said chassis having first and second end portions and a central portion extending therebetween, and including a first covering layer having a first surface intended to face towards a user and a second surface intended to face away from the user in use, wherein said waist belt is attached to the chassis so that a first belt end section and second belt end section of the waist belt extending on each side of the first end portion of the chassis for securing to each other around a wearer of the article, and wherein said second end portion of the chassis includes a fastening device for securing the second end portion of the chassis to the belt,
wherein the waist belt has an inner surface facing the wearer during use and an outer surface facing away from the wearer during use, said first belt end section is provided with a fastening component and said second belt end section is provided with a mating fastening component, wherein each one of the fastening component and the mating fastening component comprises a first mechanical connector structure and a second mechanical connector structure, each mechanical connector structure being capable of forming a mechanical interconnection with said other mechanical connector structure, and being incapable of forming a mechanical interconnection with an identical structure, whereby said fastening component and said mating fastening component are mechanically connectable via said first mechanical connector structure and said second mechanical connector structure to form an interconnection between said first transverse belt end section and said second transverse belt end section for securing to each other around a wearer of the disposable absorbent article.

25. A disposable absorbent article comprising a chassis and a waist belt, said chassis having first and second end portions and a central portion extending therebetween, and including a first covering layer having a first surface intended to face towards a user and a second surface intended to face away from the user in use, wherein said waist belt is provided separate from said chassis, and wherein said second end portion of the chassis includes a fastening device for securing the second end portion of the chassis to the belt,
wherein the waist belt has an inner surface facing the wearer during use and an outer surface facing away from the wearer during use, said belt comprising a first belt end section provided with a fastening component and a second belt end section provided with a mating fastening component, wherein each one of the fastening component and the mating fastening component comprises a first mechanical connector structure and a second mechanical connector structure, each mechanical connector structure being capable of forming a mechanical interconnection with said other mechanical connector structure, and being incapable of forming a mechanical interconnection with an identical structure, whereby said fastening component and said mating fastening component are mechanically connectable via said first mechanical connector structure and said second mechanical connector structure to form an interconnection between said first transverse belt end section and said second transverse belt end section for securing to each other around a wearer of the disposable absorbent article.

26. The disposable absorbent article according to claim 24, wherein said fastening component is arranged on the inner surface of said first belt end section and said mating fastening component is arranged on the outer surface of said second belt end section.

27. The disposable absorbent article according to claim 24, wherein said first mechanical connector structure and the second mechanical connector structure of said fastening component is arranged in a first pattern and the first mechanical connector structure and the second mechanical connector structure of said mating fastening component is arranged in a second pattern, wherein said first pattern and said second pattern are formed so that at least a portion of the first mechanical connector structure of said fastening component mirrors at least a portion of the second mechanical connector structure of said mating fastening component and at least a portion of the second mechanical connector structure of said fastening component mirrors at least a portion of the first mechanical connector structure of said mating fastening component.

28. The disposable absorbent article according to claim 27, wherein the entire first pattern is a mirror image of the entire second pattern.

29. The disposable absorbent article according to claim 24, wherein the size and orientation of the fastening component on the first belt end section, as seen in the direction x and direction y, corresponds to the size and orientation of the mating fastening component on the second belt end section, as seen in the direction x and direction y.

30. The disposable absorbent article according to claim 24, wherein said second belt end section comprises a plurality of mating fastening component arranged spaced apart on the outer surface of said second belt end section, each mating fastening component comprising first mechanical connector structure and second mechanical connector structure, so that said fastening component and any one of the plurality of said mating fastening components are mechanically connectable via said first mechanical connector structure and said second mechanical connector structure to form an interconnection between said belt end section and said second belt end section for securing to each other around a wearer of the disposable absorbent article.

31. The disposable absorbent article according to claim 24, wherein one of said first and second mechanical connector structures comprises hooks, and the other of the first and the second connector structures comprises loops.

32. The disposable absorbent article according to claim 24, wherein one of said first and second mechanical connector structures comprises a first type of hooks, and the other of the first and the second mechanical connector structures comprises a second type of hooks, said first type of hooks having a different hook characteristics than said second type of hooks.

33. The disposable absorbent article according to claim 24, wherein said first mechanical connector structure is provided by a first connector material and said second mechanical connector structure is provided by a second connector material.

34. The disposable absorbent article according to claim 33, wherein said second connector material is attached to said first connector material.

35. The disposable absorbent article according to claim 33, wherein any one of said fastening component and said fastening component comprises said first connector material onto which intermittent pieces of said second connector material are attached.

36. The disposable absorbent article according to claim 33, wherein any one of said fastening component and said mating fastening component comprises a first connector material onto which is attached a second connector material in which through holes are formed, such that the first connector structure of the first connector material is accessible via the through holes in the second connector material.

37. The disposable absorbent article according to claim 33, wherein any one of said fastening component and said mating fastening component comprises a carrier material, onto which said first and/or second connector material is attached.

38. The disposable absorbent article according to claim 33, wherein any one of said fastening component and said mating fastening component is a single connector material comprising a backing from which the connector structures extend.

39. The disposable absorbent article according to claim 24, wherein each one of the fastening component and mating fastening component having a length, and a longitudinal central axis being defined along said length, wherein said pattern formed by said first and said second connector structures is such that at least a portion of said first structure on one side of said longitudinal central axis, mirrors a portion of said second structure on said other side of said longitudinal central axis.

40. The disposable absorbent article according to claim 39, wherein a transversal central axis is defined along said width of each fastening component and mating fastening component, wherein, said pattern formed by said first and second connector structures is such that at least a portion of said first structure on one side of said transverse central axis, mirrors a portion of said second structure on said other side of said transverse central axis.

41. The disposable absorbent article according to claim 39, wherein said at least one portion of said first mechanical connector structure, located on one side of said longitudinal central axis and on one side of said transversal central axis; mirrors a portion of said second mechanical connector structure on said other side of said longitudinal central axis and another portion of said second mechanical connector structure on said other side of said transversal central axis.

42. The disposable absorbent article according to claim 41, wherein only one out of said first and second mechanical connector structures is located on one side of said longitudinal central axis.

43. The disposable absorbent article according to claim 24, wherein any one of the first belt end section and the second belt end section comprises an elasticised region.

44. The disposable absorbent article according to claim 24, wherein the waist belt is an extension belt product for the disposable absorbent article.

45. The disposable absorbent article according to claim 24, wherein an intermediate belt section is arranged between said first belt end section and said second end belt section to form a waist belt intended for use with the disposable absorbent article.
